(19) United States
(12) Patent Application Publication
(10) Pub. No.: US 2007/0185735 A1
(43) Pub. Date: Aug. 9, 2007

(54) SYSTEMS AND METHODS FOR PROVIDING A CUSTOMIZED MEDICAL HEALTH CARE MONITORING SYSTEM

(76) Inventor: Robin Peng, Sandy, UT (US)

Correspondence Address: BAKER & ASSOCIATES PLLC, 470 EAST NINTH AVENUE, SALT LAKE CITY, UT, 84103, US

(21) Appl. No.: 11/670,977

(22) Filed: Feb. 3, 2007

Related U.S. Application Data
(60) Provisional application No. 60/765,106, filed on Feb. 4, 2006.

(51) Int. Cl. G06Q 10/00; A61B 5/00

(52) U.S. Cl. 705/2; 600/300

(57) ABSTRACT

The present invention relates to systems and methods for providing a customized health care monitoring system. One embodiment of the present invention relates to providing individuals with a health care monitoring system that specifically accommodates their health care needs. The method includes receiving health related information, including but not limited to age, weight, disease status, etc. The health related information is then correlated with specific health related devices that are necessary to measure particular aspects of an individual's health. The correlated health related devices are then automatically displayed to the user. The method may optionally include providing the opportunity for a user to manually select additional health related devices which may or may not be recommended based on the received health related information. The method then produces a product in which the various health related devices are configured to operate together.

### CROSS-REFERENCES TO RELATED APPLICATIONS

The present invention claims priority on earlier U.S. Provisional Application 60/765,106, filed Feb. 3, 2006, and incorporates said earlier Application herein by reference in its entirety.

### FIELD OF THE INVENTION

The invention generally relates to a method of doing business. In particular, the present invention relates to systems and methods for providing a customized health care monitoring system.

### BACKGROUND OF THE INVENTION

Due to rising health care costs and increased public availability of medical information, individuals have begun to monitor their personal health independent of medical professionals. A large number of households currently include at least one health value measuring device such as a scale, blood pressure monitor, heart rate monitor, or body fat analyzer. These devices are used to measure at least one value related to an individual's overall health. For example, a scale is used to measure the body weight of a user. Body weight is often tracked to determine if weight is gained or lost over a particular time period. Likewise, weight may be used to self diagnose degrees of obesity which are known to lead to a variety of health related problems. In addition to elective health monitoring, many individuals are required to measure certain health values as a result of one or more medical conditions. For example, a diabetic must monitor his or her insulin levels to determine when it is necessary to ingest or inject supplemental insulin. Likewise, certain heart related conditions require that a patient regularly monitor his or her blood pressure.

In certain circumstances, medical professionals may be required to routinely measure one or more health values to ensure that a patient is healthy. Often, patients that require consistent measurements must be located in a hospital or medical care facility to allow medical professionals to analyze the values. For example, in certain individuals, rapid increases or decreases in blood pressure may indicate a severe medical condition, which must be treated immediately. If a patient is disabled mentally or physically it may not be practical to accurately and reliably perform these measurements. To address this problem, various companies have designed devices and systems to allow for the remote monitoring of patients. These systems allow participants to measure their values and report them to medical professional via some form of communication device. This enables the medical professional to remotely review the values to determine if further intervention is necessary.

However, the existing systems fail to overcome many of the problems encountered in pursuit of accurate, convenient medical value measurement. Participants and patients are often still required to manually measure one or more values. Participants who are mentally or physically incapacitated will not be able to utilize this type of system because of an inability to operate the measurement equipment. In addition, participants may then be required to record the measured values and transmit information to the doctor. These tasks may also be problematic for certain disabled individuals.

In addition, conventional remote health monitoring systems are directed exclusively for severely unhealthy individuals. These systems are designed exclusively to transmit information to a medical professional for review. Therefore, there is no local interpretation of the measured health values. The only information provided to the patient is the individual measurements of each particular device.

Likewise, conventional systems are prohibitively expensive. Each of the measuring devices is often packaged independently of the overall system, thereby requiring independent power sources, user interfaces, and transmission systems. Thus, a user may be required to operate two or more measurement devices in order to provide the necessary health values for proper assessment. These devices are packaged in a manner that prevents or impedes their disposal in a particular tight location, often requiring independent storage. Further, conventional monitoring systems require an operator to install the communication link between the patient and the medical monitoring professional.

Therefore, there is a need in the industry for a medical monitoring system that may be effectively utilized by both healthy and unhealthy individuals, minimizes physical space, minimizes overall cost, and is capable of reliably recording and monitoring health care value information.

### SUMMARY OF THE INVENTION

The present invention relates to systems and methods for providing a customized health care monitoring system. One embodiment of the present invention relates to providing individuals with a health care monitoring system that specifically accommodates their health care needs. The method includes receiving health related information from a user. The health related information may include any type of information relating to health including but not limited to age, weight, disease status, etc. The health related information may be received via any form of communication including voice, data, etc. The health related information is then correlated with specific health related devices that are necessary to measure particular aspects of an individual's health. For example, if an individual provides health information that includes a weight that is significantly higher than normal for his or her height, a scale may be automatically correlated. Likewise, if an individual is at risk of sudden conditions, an emergency response button could be automatically correlated. The correlated health related devices are then automatically displayed to the user. The method may optionally include providing the opportunity for a user to manually select additional health related devices which may or may not be recommended based on the received health related information. The method then produces a product in which the various health related devices are configured to operate together.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

The following description of the invention can be understood in light of the Figures, which illustrate specific aspects of the invention and are a part of the specification. Together with the following description, the Figures demonstrate and explain the principles of the invention. The Figures presented in conjunction with this description are views of only particular—rather than complete—portions of the systems and methods of making and using the port system according to the invention. In the Figures, the physical dimensions may be exaggerated for clarity. The same reference numerals in different drawings represent the same element, and thus their descriptions will be omitted.

FIG. 1 illustrates a perspective view of one embodiment of a customized product produced in accordance with embodiments of the present invention;

FIG. 2 illustrates a flow chart of a method for producing a customized health care monitoring system in accordance with one embodiment of the present invention; and

FIG. 3 illustrates a system for producing a customized health care monitoring system in accordance with one embodiment of the present invention.

### DETAILED DESCRIPTION OF THE INVENTION

With reference now to the drawings, the preferred embodiment of the application tool is herein described. It should be noted that the articles “a”, “an” and “the”, as used in this specification, include plural referents unless the content clearly dictates otherwise.

The present invention relates to systems and methods for providing a customized health care monitoring system. One embodiment of the present invention relates to providing individuals with a health care monitoring system that specifically accommodates their health care needs. The method includes receiving health related information from a user. The health related information may include any type of information relating to health including but not limited to age, weight, disease status, etc. The health related information may be received via any form of communication including voice, data, etc. The health related information is then correlated with specific health related devices that are necessary to measure particular aspects of an individual's health. For example, if an individual provides health information that includes a weight that is significantly higher than normal for his or her height, a scale may be automatically correlated. Likewise, if an individual is at risk of sudden conditions, an emergency response button could be automatically correlated. The correlated health related devices are then automatically displayed to the user. The method may optionally include providing the opportunity for a user to manually select additional health related devices which may or may not be recommended based on the received health related information. The method then produces a product in which the various health related devices are configured to operate together.

The following terms are defined as follows:

Health care monitoring system—A device that is capable of monitoring at least two data values related to an individual's health. These values may be displayed and correlated together to make determinations as to a particular user's health status. Likewise, these data values may be tracked to assess health related progress.

Health related information—Any type of information related to an individual's health including numerical values, images, sounds, samples, etc. Health related information includes but is not limited to height, weight, blood pressure, heart rate, insulin level, blood analysis, X-ray images, audible cough, hair sample, etc.

Health related devices—A device that is in communication with a health care monitoring system. Health related devices may measure health related data values and transmit information to the health care monitoring system. In addition, a health related device may provide a health related function such as an emergency response button. Other health related devices include but are not limited to a weight scale, height measurement, a thermometer, a digital stethoscope, a blood pressure cuff, a pulse oximeter, an ECG/rhythm strip, a glucose meter, and a first aid kit.

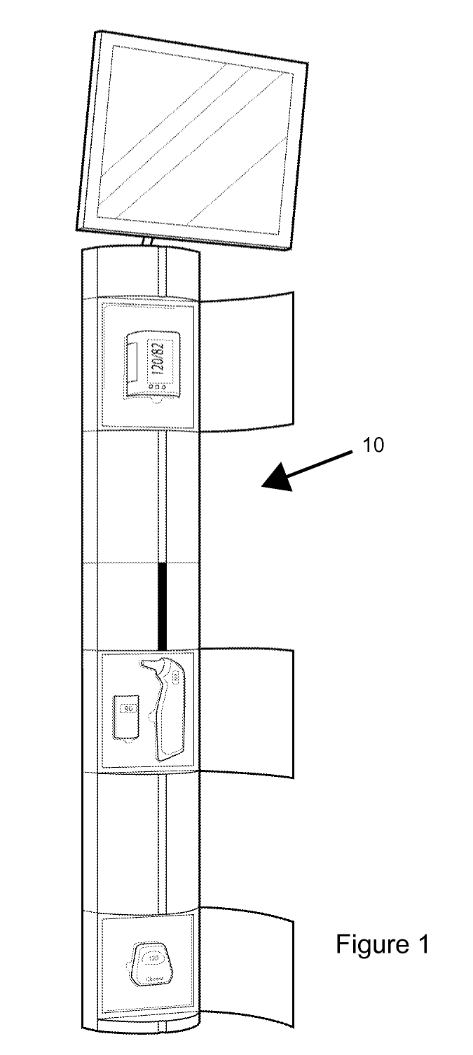

Reference is initially made to FIG. 1, which illustrates a perspective view of a product 10 produced in accordance with one embodiment of the present invention.

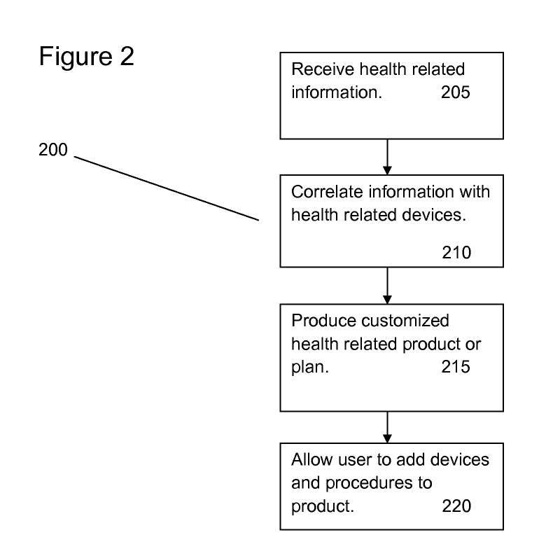

Reference is next made to FIG. 2, which illustrates a method for producing a customized health care monitoring system in accordance with one embodiment of the present invention, designated generally at 200. The method initially includes the act of receiving health related information, 205. The receipt of the health related information may be over any form of communication format including data, voice, etc. The health related information is then correlated with health related devices, act 210. This correlation utilizes an algorithm based on medical knowledge regarding whether a particular patient needs to monitor one or more health related values. A customized compatible product or plan of action (both refered to as “product” in this Application and the appended claims) is then produced, act 215 which includes all of the correlated health related devices. An optional act 220 may be included to allow participants to manually select additional health related devices to be included in the customized compatible product.

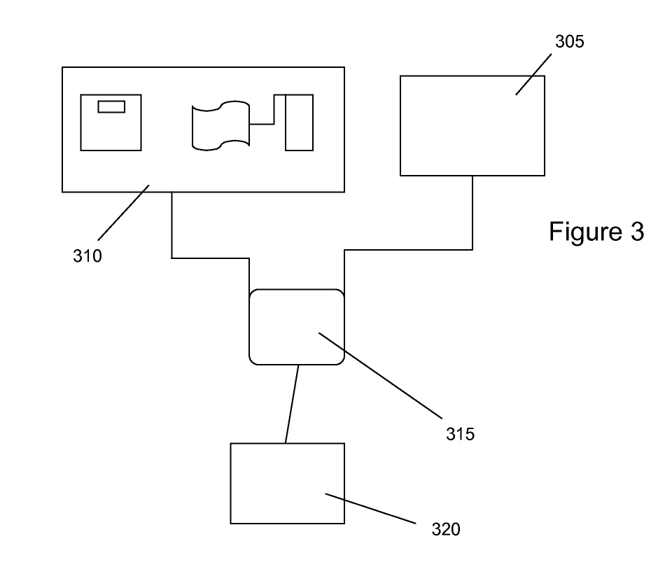

Reference is next made to FIG. 3, which illustrates a system for producing a customized health care monitoring system in accordance with one embodiment of the present invention, designated generally at 300. The system 300 further includes a user input 305 which allows users to input health related information. Alternatively, the user input could be configured to automatically enter health related information based on other information sources. For example, a DMV database may be used to generate default values for date of birth, height, weight, etc. The system further includes a product interface 310 configured to analyze the health related information from the user input 305 and a processor 315 to properly correlate it with particular health related devices based on an algorithm. The algorithm utilizes correlation data and device data which may be stored remotely or locally. Information about the correlated health related devices is communicated to a manufacturing facility 320 which produces a customized compatible product in which all of the correlated health related devices are included. Various software and firmware adjustments may be required such that the customized compatible product properly utilizes all of the health related devices.

It should be noted that this method may be implemented in a nursing or assisted living situation, a health care facility or a patient's personal physician, where the database used in step 305 could be a proprietary database within the living system and residents and/or patients could self test themselves at prescribed intervals. The system then takes over a portion of the “manufacturing” 320 by reacting to a resident's needs based on measures medical values and personal choice of the resident.

Although the present invention has been described with reference to preferred embodiments, numerous modifications and variations can be made and still the result will come within the scope of the invention. No limitation with respect to the specific embodiments disclosed herein is intended or should be inferred.

What is claimed is:

1. A method for producing a customized health care monitoring system comprising the acts of:
a. receiving health related information from a user;
b. correlating the received health related information with health related devices based upon a known correlation algorithm; and
c. producing a customized compatible product that includes the correlated health related devices.

2. The method of claim 1, wherein the act of correlating the received health related information with health related devices includes determining if a particular attribute is above or below the accepted healthy range.

3. A system for producing a customized health care monitoring system comprising:
a. a user input system configured to allow a user to enter personal health related information, wherein personal health related information includes values typically recorded in a medical history, wherein the user input system is configured to transmit the personal health related information;
b. an interface which include a data receiving device for receiving data from the user input system, at least one data storage device for storing health related information, wherein the interface utilizes data from the at least one data storage device to correlate the personal health related information with one or more health related devices based upon an algorithm; and
c. a manufacturing facility in communication with the interface such that the correlated health related devices can be manufactured into a customized compatible product.

4. The system of claim 3, wherein the data utilized by the interface includes recently published research that may affect whether a particular health related device should be correlated to particular health attributes.

5. The system of claim 3, the manufacturing facility being an assisted living facility.

6. The system of claim 3, the manufacturing facility being an health care facility.

7. The system of claim 3, the manufacturing facility being a patient's personal physician.
